(19) United States
(12) Patent Application Publication
(10) Pub. No.: US 2018/0161502 A1
(43) Pub. Date: Jun. 14, 2018

(54) INSERTION SYSTEM AND METHOD

(71) Applicant: The University of Sydney, New South Wales (AU)

(72) Inventors: Ralph NANAN, Sydney (AU); Philip BOUGHTON, Sydney (AU); Jinman KIM, Sydney (AU); Alistair MCEWAN, Sydney (AU); Graham BROOKER, Sydney (AU); Jonathan BRUCK, Sydney (AU)

(73) Assignee: The University of Sydney, New South Wales (AU)

(21) Appl. No.: 15/736,094

(22) PCT Filed: Jun. 15, 2016
(86) PCT No.: PCT/AU2016/050497

§ 371 Date: Dec. 13, 2017

(30) Foreign Application Priority Data
Jun. 15, 2015 (AU) 2015902271

(51) Int. Cl. A61M 5/20; A61M 5/42; A61M 5/32; A61B 5/15; A61B 5/00

(52) U.S. Cl. CPC A61M 5/20; A61M 5/427; A61B 5/489; A61B 5/15; A61M 5/3287

(57) ABSTRACT

A needle insertion device configured to insert a needle into a lumen of a vessel of a subject, the insertion device comprising an insertion mechanism configured to insert the needle into the lumen of the vessel at high speed, in accordance with one or more predetermined parameters.

### FIELD OF THE INVENTION

The present invention relates to the insertion of a needle or other similar piercing device into a lumen of a vessel. In a preferred form, the present invention relates to the insertion of a needle and an associated cannula assembly into a vessel, although aspects of the described invention may be applicable to the insertion of a needle or other piercing device for reasons other than cannula insertion.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

Cannulation is the process of inserting a small plastic tube or cannula into the lumen of a blood vessel after an initial puncture with a needle or trochar. Peripheral venous cannulation is used to deliver fluids and medications, sample blood and monitor the patient condition and as such it is one of the most common invasive procedures in modern medicine. Cannulation can be performed on both human and non-human animal patients.

Although common, cannulation can present a challenge in certain circumstances, most notable in specific sections of the population, such as the obese, premature neonates, geriatric patients or in patients requiring immediate venous access in life threatening situation. In these situations, small, fragile veins, vein mobility or poor surrounding connective tissue due to oedema or fat tissue can complicate access.

If the clinician requires multiple attempts to insert a cannula, the experience can be painful and distressing for the subject. This affects the trust relationship between the patient and the health care provider; can lead to delayed treatment; increased hospital time; and may also affect the clinician's confidence.

These problems are exacerbated in situation where cannulation is performed in less than ideal contexts; such as by insertion by less experienced personnel; insertion outside hospital environments; or in time critical situations, such as might be encountered by the defence forces, paramedics or emergency services personnel.

The conventional technique for venous cannulation involves allowing the veins to dilate to make location and insertion of the needle more straightforward. Dilation is commonly achieved by raising the pressure slightly (e.g. by applying a tourniquet) to make the veins easier to find and more stable when cannulating. Light tapping and application of an alcohol swab can also assist with vein dilation. For more difficult situations, a warm damp towel can be used to induce local vasodilation, or a strong light can be used to see the dark veins as the light passes either through the tissue of the patient or into the surrounding tissue.

More recently imaging devices have been used to assist in vessel identification. Two examples of such devices that use near infrared light for imaging are the Accuvein™ and VeinViewer™ devices. In other circumstances, a conventional cart based ultrasound can be used to find a vein and assess the depth and straightness.

Autonomous cannulation systems, such as the VascuLogic™ VenousPro™ and the Veebot™ system have also been proposed. However, these devices are bench-top solutions that use a highly sophisticated custom robotic arm and are not suited for the most common clinical situations in which cannulation is performed.

Accordingly there is a need for devices and methods that can assist in the process of in needle and/or cannula insertion and which address one or more of the drawbacks of the above-mentioned systems.

### SUMMARY OF THE INVENTION

In a first aspect, the present invention provides a needle insertion device configured to insert a needle into a lumen of a vessel of a subject, the insertion device comprising:

an insertion mechanism configured to insert the needle into the lumen of the vessel at high speed, in accordance with one or more predetermined parameters.

The needle insertion device can include a targeting system configured to determine said one or more parameters.

In preferred forms, the high speed is more than 10 mm/s, 20 mm/s, 30 mm/s, 40 mm/s, 50 mm/s, 60 mm/s, 70 mm/s, 80 mm/s, 90 mm/s, 100 mm/s, or more than 200 mm/s, 250 mm/s, 300 mm/s, 400 mm/s, 500 mm/s, 600 mm/s, 700 mm/s, 800 mm/s, 900 mm/s, 1000 mm/s, or more than 1500 mm/s.

In preferred forms, the high speed is less than 2000 mm/s.

In a second aspect, the present invention provides an automatic hand-held needle insertion device configured to insert a needle into a lumen of a vessel of a subject, the insertion device comprising:

an insertion mechanism configured to insert the needle into the lumen of the vessel, in accordance with one or more predetermined parameters, and

a hand-holdable portion grippable by an operator to enable manipulation of the device in use.

In one form, the needle insertion device is grippable by a single hand of the operator.

In preferred forms, the needle insertion device is weighed so that it can be single-handedly operated by the operator.

In one form, the needle insertion device weighs less than 1000 g, or less than 500 g.

Preferably the needle insertion device does not include a restraint for securing the device relative to a body part of the subject containing a portion of the vessel into which the needle is to be inserted.

In a third aspect, the present invention provides a needle insertion device configured to insert a needle into a lumen of a vessel of a subject, the insertion device comprising:

an insertion mechanism configured to insert the needle into the lumen of the vessel, in accordance with one or more predetermined parameters.

In one form, the predetermined parameters are selected and/or determined based on the subject's physical characteristics.

In one form, the device further includes a targeting system configured to determine the one or more parameters.

In one form, the targeting system includes targeting algorithm that receives as input one or more physical characteristics of the subject that the device is to be used on, and automatically determines the one or more parameters.

In one form, the targeting system includes a three dimensional imaging system, said imaging system being configured to determine the one or more parameters for insertion of the needle into the lumen of the vessel.

In one form, the targeting system determines one or more of the following parameters for insertion of the needle:
an insertion site on the tissue surface;
a target position within the lumen of the vessel;
a needle trajectory;
angle of insertion;
speed of insertion and depth of insertion.

Most preferably, the targeting system does not provide active guidance of the needle during insertion, e.g. based on feedback from the imaging system. This is enabled by the use of high speed insertion, whereby the vessel position is relatively constant throughout the insertion process. Furthermore, it is believed that use of high speed insertion minimises tissue deformation during insertion, which in turn assists in accurate placement of the needle after insertion.

The insertion mechanism can include a carriage, which in use holds the needle and is translatable with respect to one or more guides. In one embodiment, translation of the carriage may be caused by an actuator. The actuator can be of any suitable type, including: electrical, hydraulic, mechanical or pneumatic. In the primary embodiment illustrated herein, the actuator is a spring, however in other embodiments a solenoid may offer advantages.

In an embodiment that uses a spring or other stored energy actuator, the insertion mechanism can include a latch and trigger mechanism to hold the actuator in a loaded state, in which energy is stored, and release the actuator to drive the carriage. The trigger can be operated by a servo or other device for causing motion of the trigger.

The insertion mechanism is arranged so that the carriage moves over a predetermined stroke. The stroke can be fixed or variable. If variable, the stroke is preferably determined by the targeting mechanism prior to actuation of insertion. In the case that the stroke is fixed, the end point of travel of the carriage can be determined by adjusting the starting position of the carriage with respect to the lumen of the vessel. The starting position of the carriage can be determined by the targeting system.

The stroke of the carriage can be terminated by a stop. The stop may be a mechanical stop, an electro-mechanical brake or other mechanism for terminating the travel of the carriage at a desired end point. In embodiments with a variable stroke, the end point of the stroke can be set by positioning the stop.

In the case that the stroke is fixed, the position of the stop can be fixed with respect to the starting position of the carriage, so that movement of the starting point determines the location of the stop.

The carriage can include two separable carriages. A first separable carriage can be configured to carry a needle, and the second separable carriage can be configure to carry a cannula arrangement during insertion. During insertion the separable carriages move in concert. The carriages can be arranged to be separated after insertion to enable the needle and cannula to move independently.

The insertion device can include a retraction system. The retraction system is preferably arranged to retract the needle after insertion. The retraction system can include a coupling configured to engage the first separable carriage after insertion. Activation of the retraction system can thereby cause retraction of the first separable carriage and the needle carried thereon.

The insertion device can include a cannula advancement system. The cannula advancement system is preferably arranged to advance the cannula after insertion of the needle. The cannula advancement system can include a coupling configured to engage the second separable carriage after insertion. Activation of the cannula advancement system can thereby cause advancement of the second separable carriage and the cannula carried thereon. The cannula advancement system can include a modulation system arranged to modulate motion of the cannula during advancement. Modulation can take the form of rotation, or vibration or other variation in motion that is used to minimise binding of the cannula on tissues through which it is being advanced.

In a preferred form, the needle retraction system and cannula advancement system can operate in concert. In one form, they are coupled by a common drive mechanism.

The three dimensional imaging system preferably includes at least two sensor systems. Preferably the sensor systems use different sensing modalities. In one form, each of the sensor systems can operate using any one or more of the following sensing modalities:
Ultrasound
Transillumination
Near Infrared, visible or other EM radiation imaging
Thermography
Tactile/Mechanical Imaging
Optical Imaging including but not limited to Photoacoustic/Optoacoustic and optical coherence tomography.
Electrical Imaging including but not limited to Electrical/RF tomography including MRI.
Radiation Imaging including but not limited to Stereo X-Ray or CT scanning.

In some embodiments the imaging system could also be used with various “contrast enhancing” sensing techniques including, elastography, contrast agents, physiological gating e.g. ECG or pulse gated imaging and manipulations such as cuff-based occlusions to enhance vessels during imaging.

In some embodiments the imaging system determines an ideal insertion site based on tissue properties and the subject's anatomy.

In some embodiments the active guidance includes operating instructions that can guide the operator of the needle insertion device to move the device to the ideal insertion site.

In preferred forms, the instructions can include one or more of visual, auditory and tactile feedback instructions to guide the operator to aim the device.

In a preferred form, the sensor systems include a Near Infrared camera and an ultrasound imaging system.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIGS. 1A and 1B illustrate bottom and top perspective views of a needle insertion device according to an embodiment of the present invention.

FIG. 2 is a bottom perspective view of the insertion device of FIG. 1 with its housing illustrated in transparent form to reveal details of the insertion mechanism.

FIG. 2A is a bottom perspective view of an insertion device in accordance with another embodiment of the present invention.

FIG. 3 is a perspective view from the rear of the need insertion device with the rear wall of the housing wall to better illustrate a portion of the insertion mechanism.

FIG. 4 is a cross-sectional view along line 4-4 of FIG. 3.

FIG. 5 is a cross-sectional view along line 5-5 of FIG. 3.

FIGS. 6A and 6B illustrates two views of the insertion mechanism of the insertion device of FIGS. 1A and 1B. In particular, FIG. 6A shows the rear wall of the housing to which a part of the guide is mounted, whereas FIG. 6B has this detail removed.

FIG. 7 shows additional detail of the insertion mechanism with the needle assembly omitted.

FIGS. 8A and 8B show two views of the device of FIG. 1 with the carriage retracted into its loaded position and latched by the latch and trigger mechanism prior to actuation. FIG. 8A shows the housing in transparent form whereas FIG. 8B does not show the housing.

FIG. 9 is a top perspective view of the cannula advancement system and needle retraction system and their associated endstops prior to advancement of the cannula and withdrawal of the needle.

FIG. 10 shows the insertion mechanism with the cannula partially advanced, and the needle partially retracted.

FIG. 11 is a flowchart illustrating a method of performing image processing that may be performed by the sensor system according to an embodiment of the present invention.

FIG. 12 is a flowchart illustrating a method of operation of the insertion mechanism in inserting a needle into a vessel after targeting.

### DETAILED DESCRIPTION OF THE EMBODIMENTS

A preferred embodiment of the present invention will now be described in connection with an insertion device configured to insert a needle 112A and an associated cannula 112B (together a “needle and cannula assembly 112” in this description) into a blood vessel. As will be appreciated, the person skilled in the art could modify the present device for insertion of other types of device, in particular other types of cannula or needle.

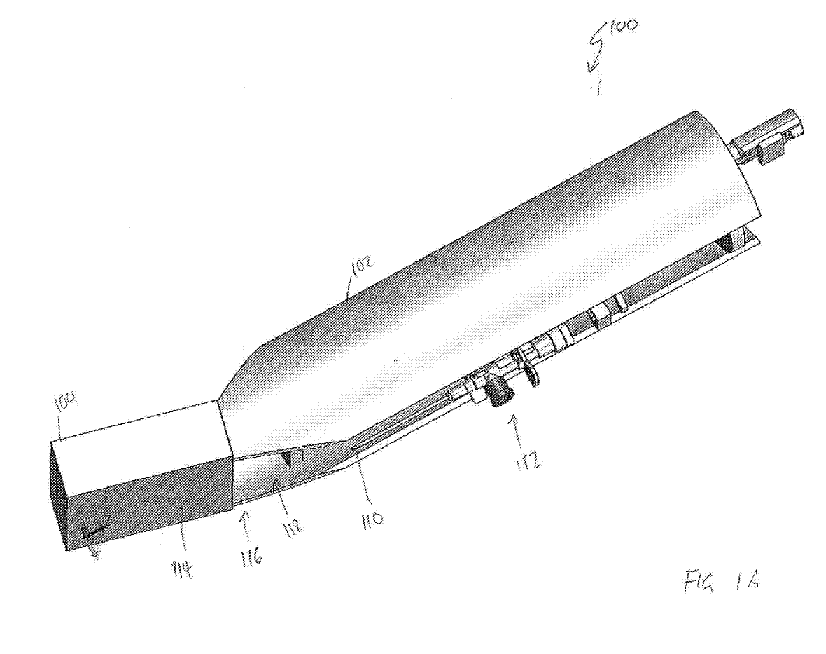

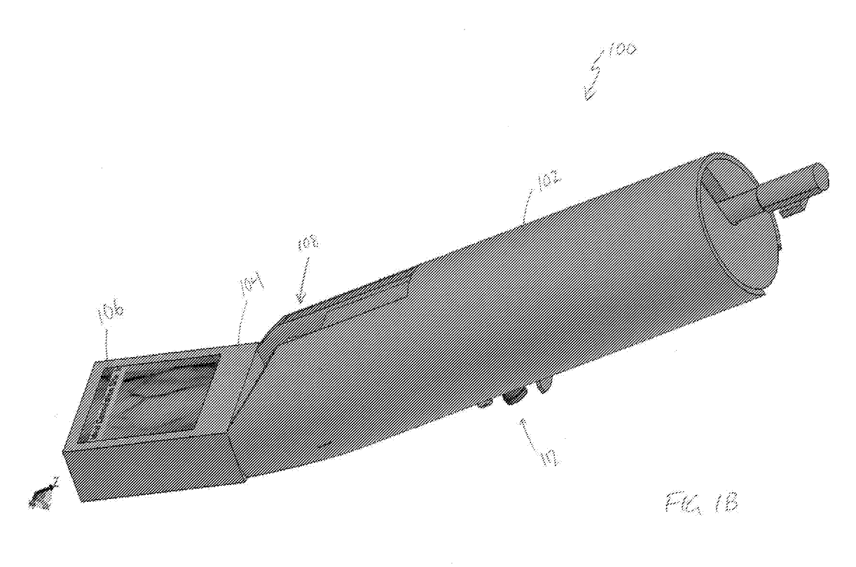

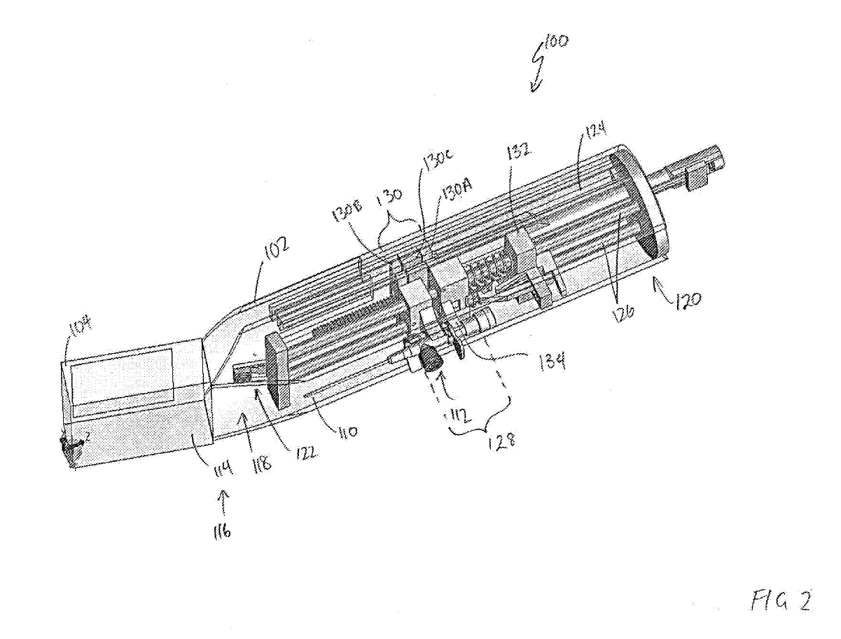

Turning firstly to FIGS. 1A and 1B which show bottom and top perspective views respectively of the insertion device 100 in overview. The insertion device 100 comprises:
a main housing 102, which contains an insertion mechanism, and can additionally house electronics, batteries and the like; and
an imaging unit 104, which houses an imaging sensor and a display 106 on which the output of the imaging system is displayed to the user.

The display can also provide user instructions and a control interface. The display 106 may be a touch screen so that it enables a user to provide control inputs to the device.

The housing 102 further includes a window 108 through which the operator can see the tip 110 of the needle 112A during use.

Preferably, the needle insertion device 100 is a hand-held device. In order to be able to be used as a hand held device, it is preferred that the device weighs less than about 1000 g, but preferably less than about 500 g. In a preferred form the needle insertion device includes a portion that is gripable by a user. Most preferably the device is able to be held and maneuvered by a user using a single hand. The user can thus use their other hand to restrain or support the subjects body part and/or control the device interface. In the embodiment shown in FIGS. 1A and 1B, the main housing 102 is grippable. Furthermore, in preferred forms the the needle insertion device does not include a restraint for securing the device to a body part of the subject. This enables the user to freely move the needle insertion device 100 to a suitable insertion site on the subject's body.

As can be seen, the needle and cannula assembly 112 is held on the underside of the body so as to be hidden from view of the patient during use. During use, the insertion device 100 is placed in contact with the patient's body such that the site at which the needle 112A is to be inserted can be viewed through window 108. The insertion device 100 is held so that the underside 114 of the imaging unit 104 as in contact with the site of insertion. The imaging sensor(s) contained within the imaging unit 104 and body are used to obtain images of the vasculature below the patient's skin to enable guidance of the device by user and control of the insertion mechanism. As will be explained in more detail below, in a preferred form, the imaging system includes multiple image sensors, each of which operates with a different imaging modality. In this example, an ultrasonic imaging system is built into the imaging building unit 106 and additionally in the infra-red camera is mounted in the distal end 116 of the housing 102. The infra-red camera can capture images in the infra-red spectrum through the enlarged opening 118 at the distal end 116 of the housing 102. Preferably the camera includes polarising filters on one or both of its light source (e.g. LEDs) and the cameras, to minimise specular reflection.

In this example, the display 106 will show to the clinician the NIR camera image directly in front of the ultrasound sensor and the ultrasound image. The positioning of the display 106 means that from the point of view of the clinician, the display appears to show a direct line of sight into the patient. This is known as “augmented reality” as the clinician will see the skin surface and additional information (the ultrasound image) overlaid on the image.

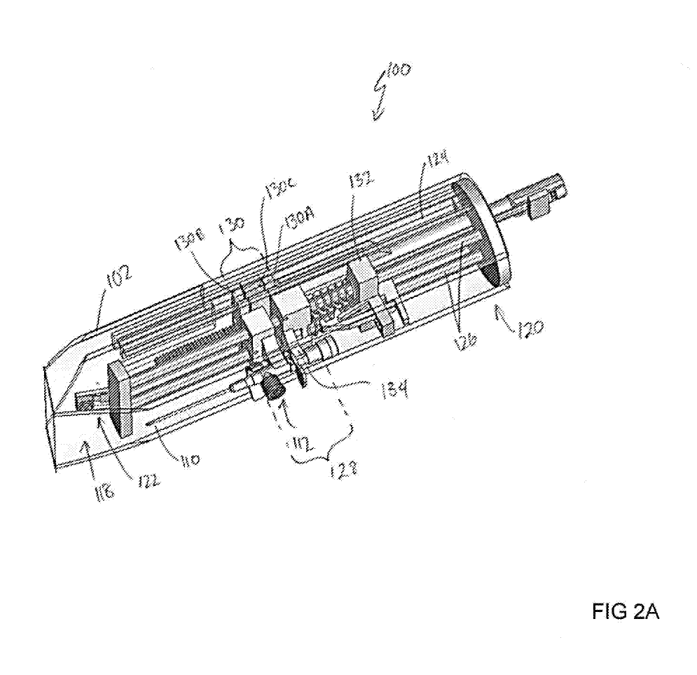

FIG. 2 shows a perspective view almost identical to that of FIG. 1A, however the housing 102 has been made transparent in order to show mechanical detail of the insertion mechanism 120 mounted within the housing 102. In this orientation, the sensor 122, in the form of an infra-red camera, which this embodiment is run by a Raspberry Pi computer system, is mounted such that it can capture images through opening 118 at the distal end 116 of the housing 102.

In order to simplify an illustration of the embodiments, components, such as actuators have been omitted from the following views. The person skilled in the art will be aware of range of actuators suitable for imparting motion to various components of the insertion device.

The insertion mechanism 120, generally comprises a guide, which in this example takes the form of a pair of rails 124 and a pair of shafts 126, along which carriage 128 is translated in use. The rails 124 and shafts 126 are additionally used to hold and control movement of end stop arrangement 130, which is used to determine the end of the stroke of the carriage 128.

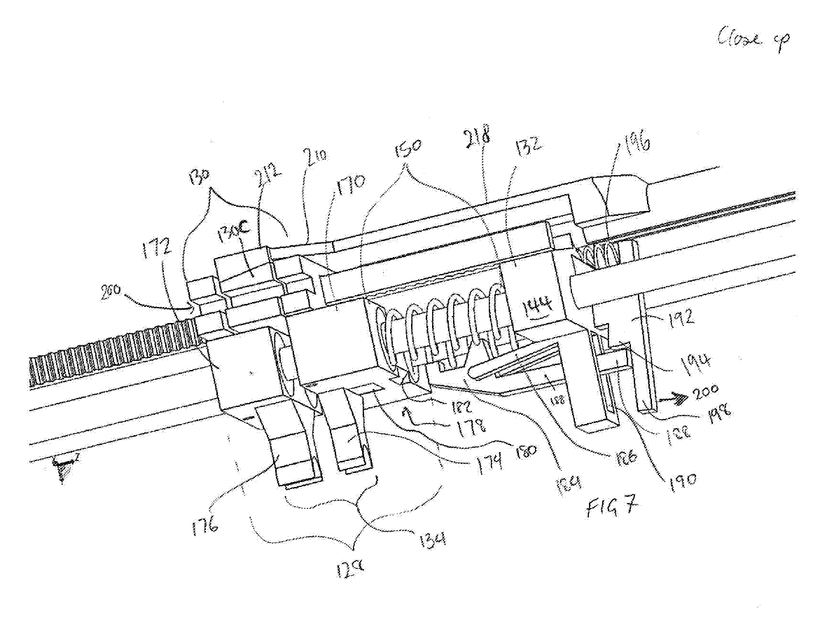

In this example, the end stop arrangement 130 comprises a pair of end stops 130A, 130B, whose functions will be described in further detail below. End stops 130A and 130B are positioned by way of an end stop carriage 130C, which extends along the housing 102 towards the proximal end of the insertion device and is rigidly fixed to the latching trigger mechanism 132. The latching trigger mechanism is used to hold the carriage 128 in a “loaded” position, in which carriage 128 is retracted prior to insertion of the needle 112A. Further detail of the latch and trigger mechanism is provided in FIG. 7.

In use, the needle and cannula assembly 112 is releasably mounted to carriage 128 by way of needle assembly holder 134. The needle and cannula assembly 112 is held in the needle assembly holder 134 with sufficient force that motion of the carriage 128 will move the needle 112A so that it pierces tissues of the patient for insertion into the lumen of a vessel. In the view of FIG. 2, the carriage 128 is shown in its deployed position so that it abuts against the end stop assembly 130. However, as will be appreciated prior to deployment, the carriage 128 is moved to a retracted loaded position at which the latch and trigger mechanism engages with the carriage 128 to hold it in place prior to actuation.

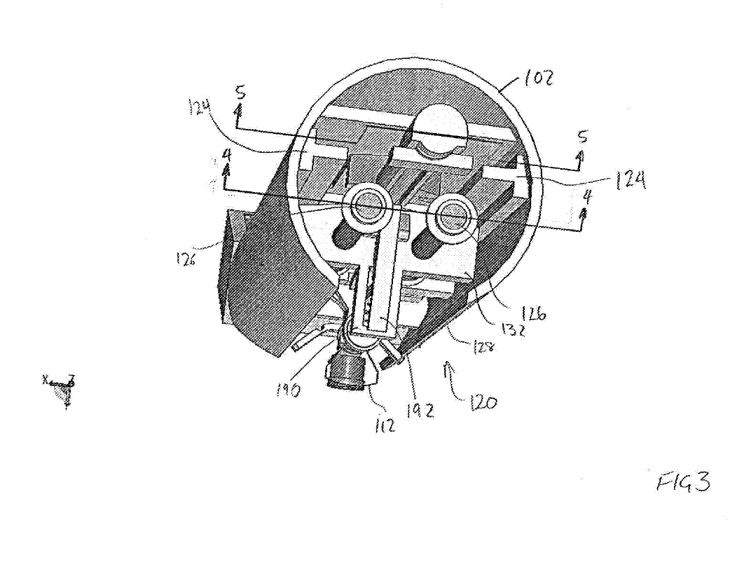

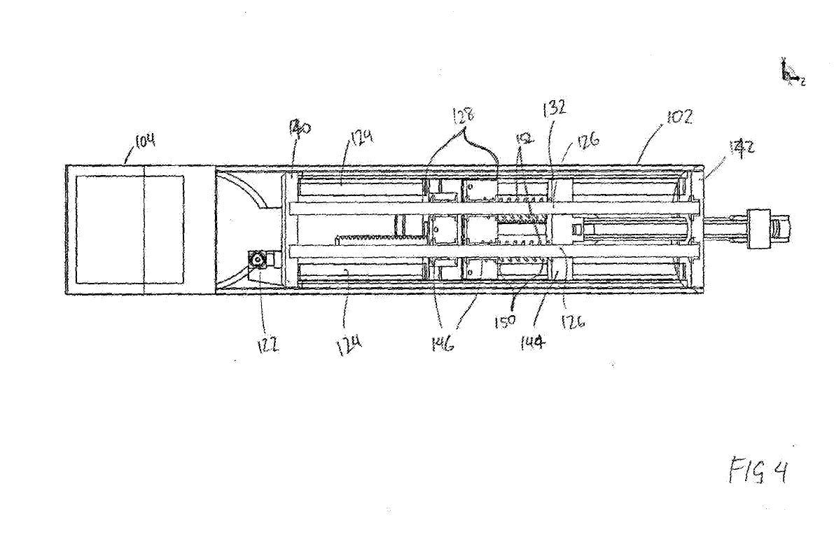

FIG. 4 shows a cross-sectional view along line 4-4 of FIG. 3 and illustrates the pair of shafts 126. The pair of shafts 126 are supported at their distal end by a front support member 140 and their proximal end by the back wall 142 of the housing 102. In this cross-sectional view, one can see a cross-section through the carriage 128 and the latch mounting block 144 of the latch and trigger mechanism 132. Because the carriage 128 is translated at high speed along the pair of rails 124, carriage 128 is provided with high speed bearings e.g. 146. In this view, the sensor 122 can also be seen.

The actuator 150 of the insertion mechanism 120 is also visible. In this example, the actuator takes the form of a pair of springs 152. The actuator acts on the carriage 128 to move it with respect to the latch mounting block 144 of the latch and trigger mechanism 132 to thereby cause movement of the needle and cannula assembly 112. Prior to insertion, the position of the latching mounting block 144, and consequently the end stop assembly 130 is fixed with respect of housing to thereby determine the location of the end of travel of the carriage, and consequently the position of the tip 110 of the needle 112A after insertion. In this example, the springs 152 are placed around the pair of shafts 126 and during loading are compressed between facing services of the latching mounting block 144 and carriage 128. In this example each spring applies a force of around 10N. However in other embodiments, springs (or other actuator mechanisms) that apply a higher total force, say between 10 and 50N could be used, whereas in other embodiments a lower force actuator, applying a force less than 20N, say down to 5N or 10N. In many uses only 0.5N force is required to pierce the skin with the needle, therefore the force applied will depend on a range of factors including but not limited to desired speed of insertion, length of stroke, mass of the components to be moved by the actuator (e.g. carriage 128 and needle and cannula assembly 112).

The extent of the pair of rails 124 can also be seen in this view. The pair of rails 124 extend the full length of the pair of shafts 126 to enable adjustment of the position of the insertion mechanism. As will be described below, the positioning of the insertion mechanism, so that the end point of the stroke of the carriage 128 is correct is determined by the targeting system.

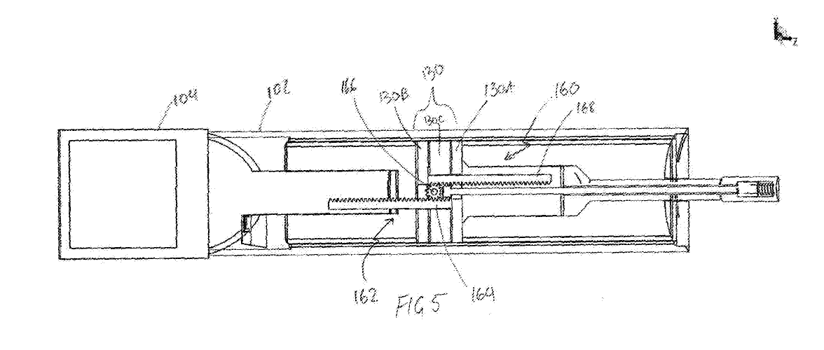

FIG. 5 shows another cross-section through the body of the insertion device, this time at line 5-5 in FIG. 3. This cross-sectional view cuts through the end stop arrangement 130, showing the pair of end stops 130A and 130B and the end stop carriage 130C as well as components of a cannula advancement sub-system 160 and a needle retraction system 162, the function of which will be described below. The needle retraction mechanism comprises a rack 164, which is mounted in a fixed relationship with respect to end stop 130A and driven by a pinion gear 166.

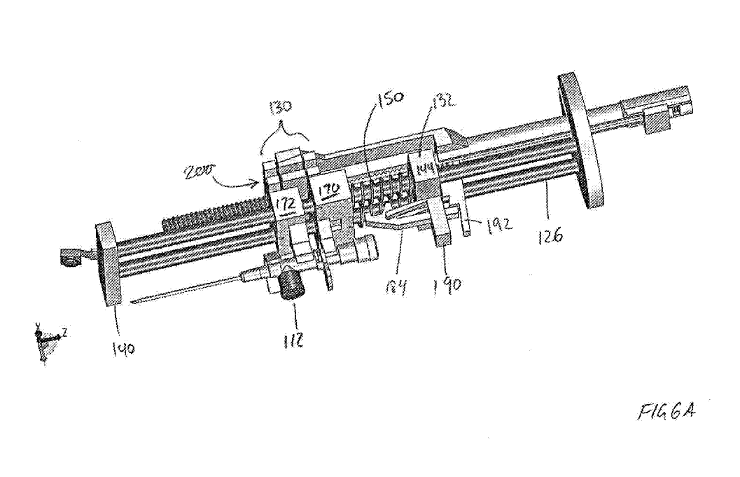

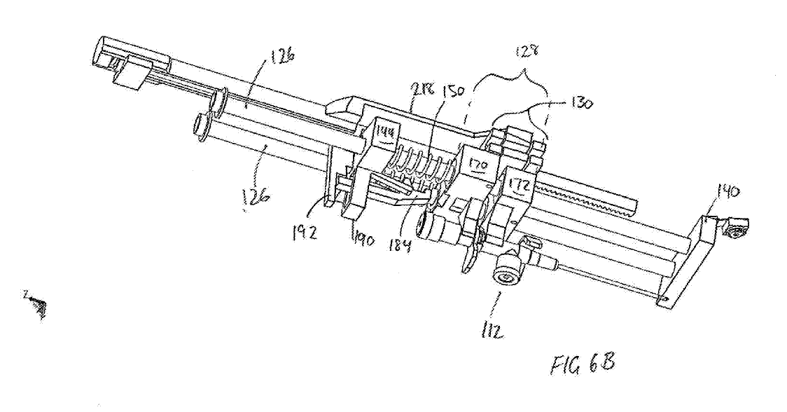

FIG. 6A. 6B and 6C show additional details of the insertion mechanism 120. As can be seen best in these views, the carriage 128 is separable into two components in the present embodiments. The first component of the carriage 128 is a needle carriage 170 the second is a cannula carriage 172. In this embodiment, the carriage 128 is separable because the needle and cannula assembly 112 has two parts (i.e. a needle 112A and a cannula 112B), which are separated after insertion so that the cannula 112B can be advanced into the lumen of the vessel and the needle 112A can then be retracted.

Because the carriage 128 is separable into two components 170 and 172, the needle assembly holder 134 also includes a needle holder 174 and a cannula holder 176. Each of the needle and cannula holders 174 and 176, respectively, in this example are formed from resilient material and comprise a pair of flexible arms defining a groove between them into which a portion of the needle 112A and cannula 112B respectively can be press fit. The resilience of the material forming the needle and cannula holders 174 and 176 serve to apply pressure to the needle 112A and cannula 112B to retain them within the needle and cannula holders 174 and 176. Prior to and during insertion, the needle carriage 170 and the cannula carriage 172 are held together so that they move in concert. The needle carriage 170 and the cannula carriage 172 can be held together mechanically by a clip, or by some other mechanism, e.g. by magnetic attraction.

As can be seen in FIG. 7, the needle carriage 170 includes a latch receiving mechanism 178 comprising recess 180 and a lead-in bevel 182, which are arranged to cooperate with and latch with the latching hook 184 of the latch and trigger mechanism 132. The latch and trigger mechanism 132 includes a mounting arm 186, to which the latching hook 184 is mounted. Mounting of the latching hook 184 is by way of a pin through the distal end of the mounting arms 186, about which the latching hook 184 pivots. At the proximal end of the latching hook 184 is a lever arm 188, which is retained within a slot 190 in the latch mounting block 144. The lever arm 188 is free to move up and down within the slot 190 and is biased towards the unlatched position by a spring mounted in the slot 190 and acting against lever arm 188. Prior to actuation of the carriage 128, the latch is held in a latched position such that the latching hook 184 is seated within the recess 180 on the needle carriage 170. The latching hook 184 is held in this position by the trigger arrangement 192. The trigger arrangement includes a locking step 194, which holds the lever arm 188 of the latching hook 184 in the latch position. The arrangement trigger 192 is mounted on a spindle or a pivot arrangement and biased towards the latched position by spring 196. In use, the lever 198 is be pulled in the direction of arrow 200 by an actuation mechanism (not shown) to release the lever arm 184 and disengage latching hook 184 to thereby actuate the movement of the carriage 128 by operation of the actuator 150. Drawing of the carriage 128 into the loaded position can be automatic, i.e. caused by operation of an actuator or by manual retraction by a user.

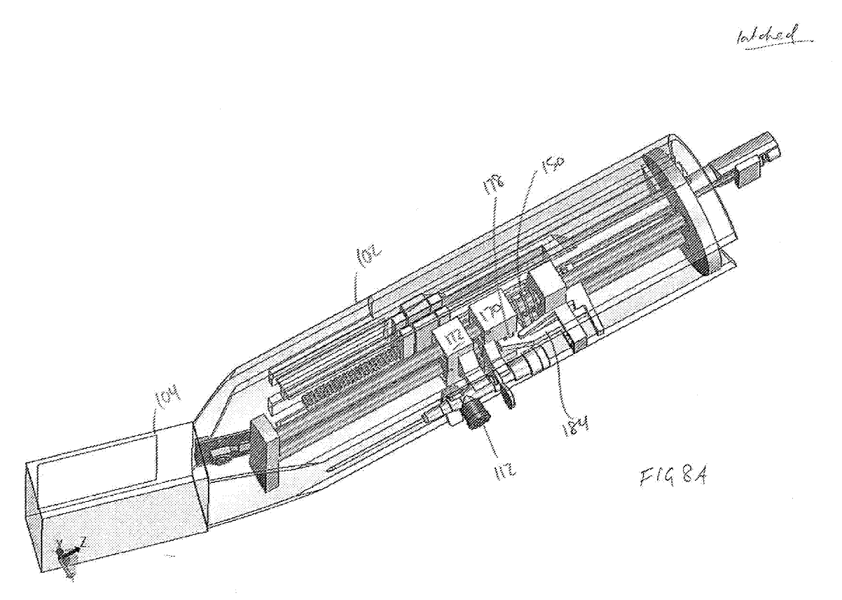

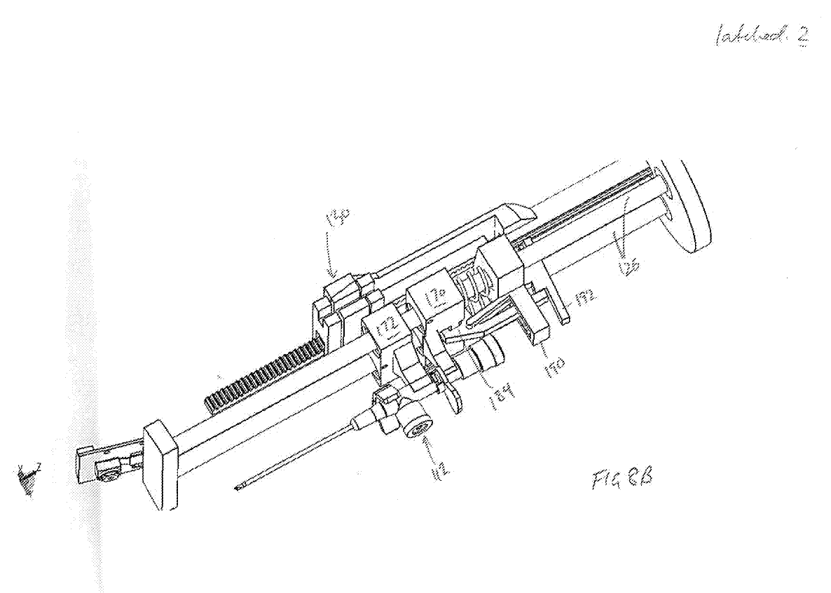

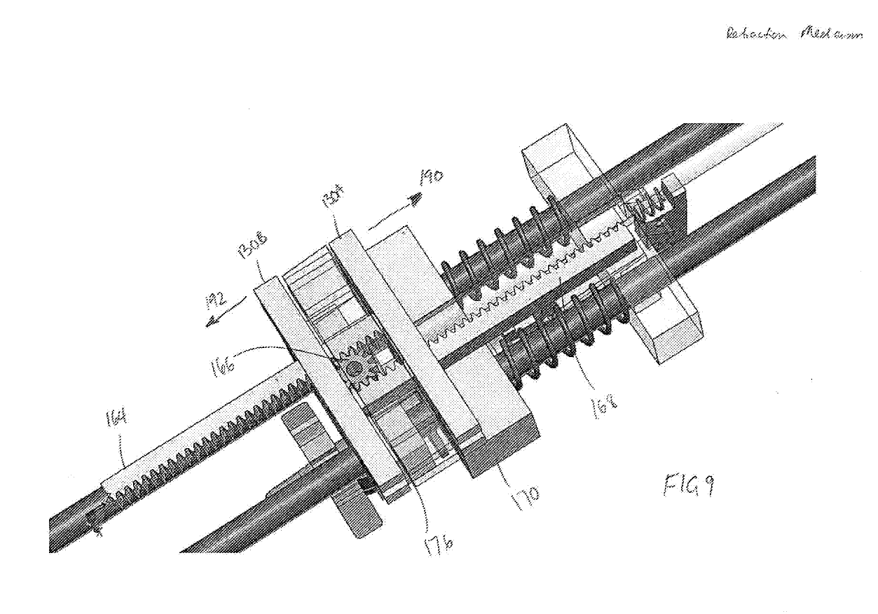

FIGS. 8A and 8B illustrate the insertion mechanism and its latch in a retracted position with the retaining hook 184 seated in the recess 180 of the needle carriage 170. In this arrangement, the actuator 150, in the form of the spring in this example is storing energy for release upon actuation. When the trigger 190 is activated by suitable actuation device e.g. a servo motor or solenoid or the like, the energy stored in the actuator 150 is released and the carriage is pushed forward at high speed along the pair of rails 126. In some embodiments the, actuator could alternatively include a solenoid or linear actuator or other mechanical mechanism capable of translating the carriage at high speed. In a preferred form, the carriage 128 moves at between 10 and 1000 mm/s. In other forms, the carriage 128 can move at a high speed of more than 10 mm/s, 20 mm/s, 30 mm/s, 40 mm/s, 50 mm/s, 60 mm/s, 70 mm/s, 80 mm/s, 90 mm/s, 100 mm/s, or more than 200 mm/s, 250 mm/s, 300 mm/s, 400 mm/s, 500 mm/s, 600 mm/s, 700 mm/s, 800 mm/s, 900 mm/s, 1000 mm/s, or more than 1500 mm/s, but preferably less than 2000 mm/s. As noted above, use of an actuator other than a spring can have certain advantages, for example actuators that can be driven in opposing directions, such as linear actuators or solenoids can additionally be used to move the carriage 128 to the loaded position instead of requiring manual loading. They may also be controllable to set the carriage stroke and/or stroke endpoint, possibly obviating the need for a mechanical end stop.

During actuation, the carriage 128 and the needle and cannula assembly 112 are pushed forward and the needle tip 110 is inserted into the patient. The carriage 128 continues moving forward until the carriage 128 reaches the deployed position in which the carriage 128 comes into contact with the end stop arrangement 130. As noted above, in this example, the end stop arrangement 130 has a pair of end stops 130A and 130B, being, a needle carriage end stop 130A and a cannula carriage end stop 130B which each stop respective portions of the carriage 128.

In some embodiments, the insertion mechanism can have a counter weight system to reduce momentum shake. In such a system, a balanced counter weight is arranged to move in the opposite direction to the carriage 128 so that the momentum of the moving components are balanced. This can help stabilise the device during key motions. Some embodiments can alternatively or additionally include an end stop arrangement 130 that does not make large impact sounds or forces during the key motions of the device. This can be achieved by having a spring or other energy absorption device, such as an elastomeric cushion or an electromechanical brake in the end stop.

After an initial insertion of the needle and cannula assembly 112 into the patient such that the tip 110 of the needle 112A is in the lumen of the target vessel, it is necessary to perform a cannula insertion process by retracting the needle and advancing the cannula. This function is performed by coupling the needle carriage 170 to the needle carriage end stop 130A, e.g. using a mechanical latch or magnetic latch or the like; and by coupling the cannula carriage 176 to the cannula end stop 130B, in a similar fashion. Then by the end stop arrangement is driven apart so as to separate the carriage components 170 and 172.

The needle advancement system comprises a pinion gear 166, which is used to drive a rack 164 that is mounted to the needle carriage end stop 130A stop. By turning the pinion gear 166, in this example when viewed from the top down, in a clockwise direction, the needle carriage end stop 130A is withdrawn in direction of arrow 190. Because only the needle 112A of the needle and cannula assembly 112 is coupled to the needle carriage 170 only the needle 112A is withdrawn in the direction of arrow 190.

The cannula advancement system comprises a gear, pinion gear 166, which engages with a rack 168 that is coupled to the cannula carriage end stop 130B. Once the cannula carriage is coupled to the cannula carriage end stop 130B and the pinion gear rotated in the clockwise direction the cannula end stop, cannula carriage 172 and hence the cannula 112B that is mounted to the cannula holder is advanced in the direction of arrow 192.

In some embodiments the cannula advancement system can include a modulation system arranged to modulate motion of the cannula during advancement. Modulation can take the form of one or more of rotation, vibration or other variation in motion that is used to minimise binding of the cannula on tissues through which it is being advanced. For example, a piezoelectric linear actuator or micromotors can be used to move the cannula transverse to the direction of insertion.

As can be seen in this example, a common pinion gear 166 is used for both the needle retraction system and the cannula advancement system. However, either separate gears or different drive systems may be employed as necessary.

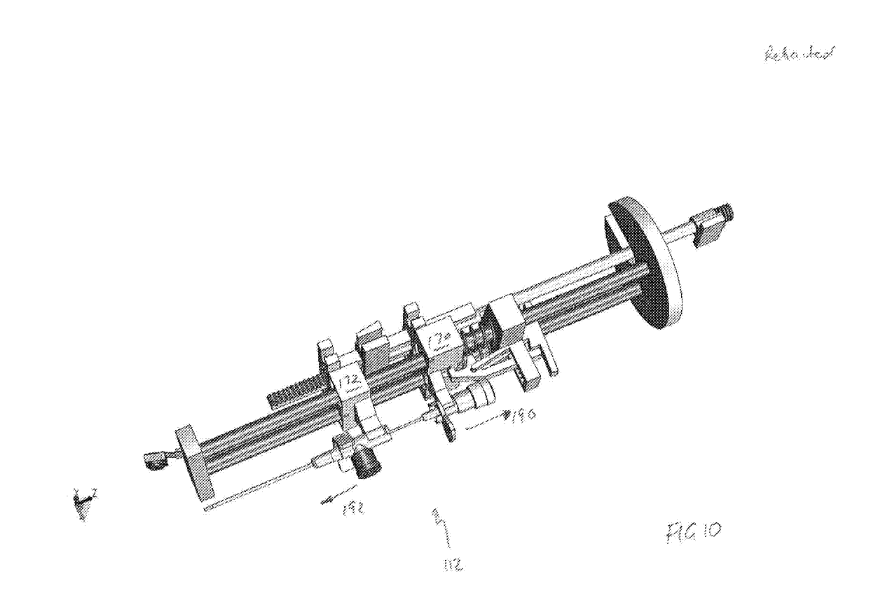

Once the cannula 112B is fully inserted it can be detached from the cannula holder and the insertion device 100 removed from the patient. As will be appreciated, the length of travel of the cannula advancement system or needle retraction system should be sufficient to enable removal of the needle from the cannula by the user without interference between the two so as to minimise patient discomfort. FIG. 10 shows the insertion mechanism with the cannula 112B advanced about half way along its stroke and the needle 112A retracted about half way towards its fully retracted position.

As noted above, the present example has a fixed stroke as determined by the separation between the end stop arrangement 130 and latch, or more precisely the latching hook 184. Thus, in order to ensure that the tip 110 of the needle 112 stops after deployment at the determined target location within the lumen of the desired vessel, the position of the end stops must be adjusted. This is performed by sliding the entire stop mechanism and consequently carriage mechanism 128 along the pair of rails 124 of the housing 102. As can be seen in FIG. 7 the pair of rails 124 are received into a groove 200 in the sides of the end stop arrangement 130 so that the end stop arrangement 130 may be accurately guided along the rails 124. The end stop arrangement 130 is moved by pushing the end stop carriage 130C forward and backwards by use of an actuating mechanism, such as a linear drive, servo motor or manual actuation by the user, and once positioned is locked into place using a clutch mechanism 210. The clutch mechanism 210 in this example is in the form of a wedge shaped lock 212 mounted on the end of a finger 218. When the block is pulled in the proximal direction it engages between the outer surface of the stop carriage 130 and the inner wall of housing 102. Thus end stop carriage 130C is effectively jammed into position such it cannot move upon actuation of the carriage 128. In order to reposition the end stop carriage 130C, e.g. to adjust the desired end point of the stroke of the carriage translation, the finger 218 is pushed in a distal direction thus releasing the binding between the lock 212, the outer surface of the end stop carriage 130C and the inside surface of the housing 102.

In this condition, the end stop arrangement 130 and the latch mounting block 144 can be translated along the pair of rails 124 and pair of shafts 126 to the required position of the next use of the device. In other embodiments, in place of the fully mechanical end stop arrangement 130, an electro-mechanical brake or other mechanism for terminating the travel of the carriage at a desired end point.

As discussed above, the preferred form of the automatic insertion device includes a targeting system. The targeting system may include a three-dimensional imaging system, which is used to determine a needle trajectory for insertion of a needle into the lumen of the vessel. The targeting system also preferably sets the operational parameters of the insertion mechanism to enable it to insert the needle in line with the determined trajectory. For example the targeting system can determine any one or more of:
a position of the end stop arrangement,
length of stroke,
position of the end of stroke (either linear or in three dimensions)
angle of insertion;
insertion location; and
time of insertion.

The determination can be performed by determining any one or more of the location or depth of veins, orientation of veins in two or three dimensions.

The three dimensional imaging system preferably includes multiple sensor systems. In one present example, the sensor systems operate using ultrasound and near infrared.

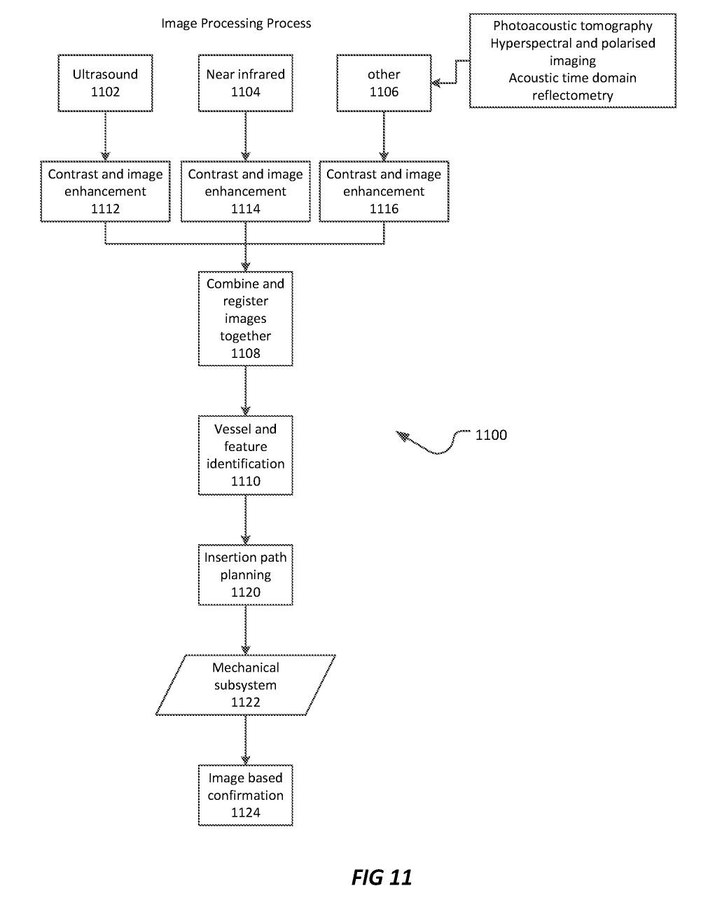

FIG. 11 illustrates a process performed by targeting system in an embodiment of the present invention.

The method 1100 begins with the acquisition of images by at least two modalities. In this example three modalities are used, being, ultrasound 1102, near infrared 1104 and a further modality 1106. The further modality could be selected from a range of imaging techniques including but not limited to photoacoustic tomography, hyperspectral and polarised imaging and/or acoustic time domain reflectometry. In other embodiments four or more imaging modalities could be used.

The ultrasound sensing system can use any type of transducer, e.g. piezoelectric or MEMS transducers. In some embodiments imaging may be a 3D real-time ultrasound. The ultrasonic sensor system can be similar to that provided by the Sonic Window™ from Analogic Corp.

Other wavelength electromagnetic radiation could be used in addition to, or an alternative to, the near infrared imaging of the illustrated embodiment. For example light in the spectrum of between about 400 nm to 2000 nm could be used. In some embodiments this may be advantageous, for example:
visible spectrum can provide information about colour,
comparing different points in the spectrum can provide information about the level of oxygenation in the vessel (due to the absorbency of oxy-versus deoxyhaemoglobin) to allow artery/vein classification,

Each image undergoes image processing, e.g. contrast and image enhancement 1112, 1114, 1116 and then are combined 1108. Combination requires care to ensure that the images are in registration with each other.

Next vessel and feature identification is performed in step 1110, followed by insertion path planning 1120. Insertion path planning involves determination of one or more of the location, angle, depth, target insertion end point, and converting this into parameters to set any one or more of the following parameters of the mechanical subsystem of the insertion device:
a position of the end stop arrangement,
length of stroke,
position of the end of stroke (either linear or in three dimensions)
angle of insertion;
insertion location; and
time of insertion.

In a preferred imaging method, the system first uses the near infrared camera to obtain images of the vasculature. In one form, NIR light at 850 nm is used. A series of candidate vessels are then ranked according to an algorithm. These algorithms are based on clinical criteria for vessel detection comprising of the length, straightness, branching and diameter of the vessel. In use, over the field of view of the NIR camera a combination of these criteria determined from the NIR image are be combined (e.g. using a weighted sum or similar prioritising scheme) and then sorted to select an optimal vessel to derive the insertion point. Once this is complete, further investigation with surface based sensors such as ultrasound, photoacoustic ultrasound or optical tomography is performed.

The surface sensors are used to determine the depth, confirm the diameter and position of the vein, examine tissue quality and determine the amount of pressure to apply from the device.

In one embodiment, processing of the ultrasound image can be performed according to the following process:
acquire an ultrasound image using the optimal insertion point from the NIR camera, e.g. from an ultrasound sensor housed within imaging unit 104;
apply a cropping algorithm to reduce the search space—cropping can be based on the analysis of a ‘line profile’ from the centre of the image from top to bottom. Another approach can be a cascade (Viola-Jones) classifier to find the approximate bounding box that contains the oval corresponding to the vessel;

apply a diffusion noise filter to remove speckle noise and to highlight the landmarks in the image;

run an edge detection algorithm based on a line preserving filter e.g., Shock Filter;

perform an iterative randomised Hough transform to look for ellipse/oval in the edge image. A statistical shape prior model (shape model can be developed using large sample of training data) can be used to create a population level circular structure to represent the average shape of a vein. Such shape can aid in the ellipse/oval detection; and
within oval, use a contrast/texture differentiating, region-based algorithm, e.g. random walker, can be applied to determine the position of the vessel.

The image processing algorithm can work on a resolution of 1px=approx. 100 μm. In the event that the NIR image has good enough field of view, the system can be used to assist with positioning of device.

In step 1110, the device may utilise precomputed image data to assist with the processing of the real time image data acquired and generated by the imaging sensors. These may be supplemented by offsite computation that returns information to the device in real time. These precomputed data may consist of pretrained image data, trained via a supervised or unsupervised machine learning and computer vision algorithms for example but not limited to neural network, using images collected from individuals with different skin types and/or vein characteristics.

In one form, the pretrained image data is manually labelled/classified with key features. These labelled key features and image data are then used to train a computer vision classifier to classify or identify key features of new images. The process of training the computer vision classifier, which involves processing a generally large amount of imaging data (for example it may go up to millions of images), is computationally expensive and is performed offsite, while passing a new single image through a trained classifier is relatively computationally cheap and is applied to the sensor platform in real time.

The location of key features are listed above, and includes bifurcation or branching of veins, straight sections of veins and larger veins. These features are to be characterized using visual attributes and each of the visual attributes may be scored. A vein location that has the highest score is recommended to the user as the ideal cannulation site. An example may be an insertion site within 3 mm of a bifurcation on a vein that is 4 mm wide and relatively straight for a distance of 12 mm.

In a preferred embodiment, an ultrasound transducer will capture depth data of the tissue to identify the vasculature. This may occlude some part of the image captured by a near infrared camera. The near infrared imaging system detailed above can accommodate for the occlusion if due to the motion of the device the camera had previously seen the area occluded by the transducer. The system may also be configured to generate an estimate for the vascular features based on the features around the ultrasound transducer and the data captured by the transducer alone.

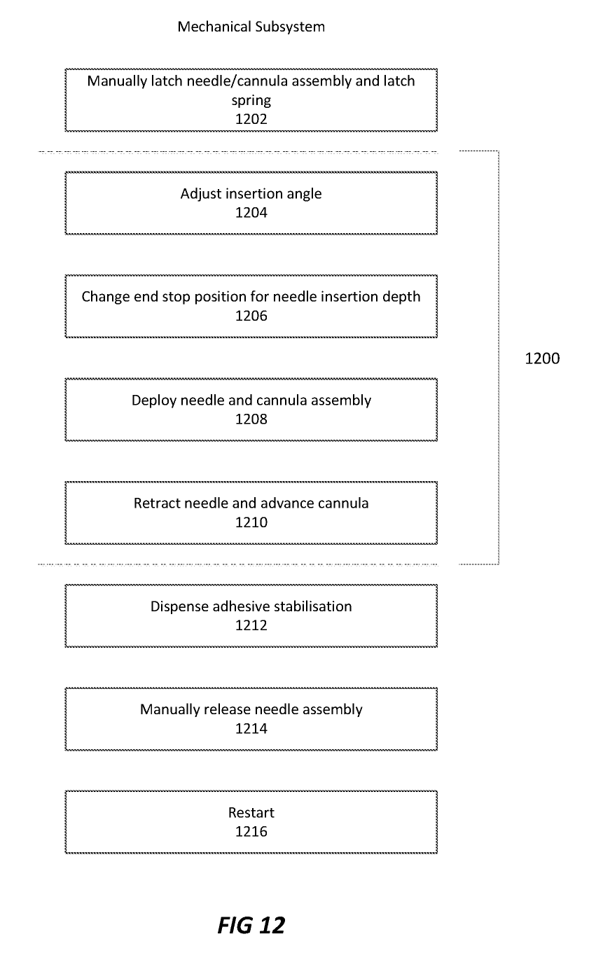

The mechanical subsystem is then adjusted and activated in step 1122. FIG. 12 illustrates a method of performing this process.

As will be appreciated before use, the user will have manually loaded the needle and cannula assembly 112 and latched the carriage 128 in the loaded position, although automated latching may be possible in other embodiments.

Once the insertion location is determined by the targeting system as described above, the system may instruct the user, e.g. using the display, audio or haptic feedback (or any combination thereof) how to adjust the positioning or pressure applied to the device prior to insertion. For example the system can indicate left, right and fore and aft, or rotational movements to the user so that they can adjust the position of the insertion device. This can be continued until the determined point of insertion coincides with the trajectory of the needle along its stroke and the orientation of the device will cause movement of the needle in the correct direction.

Next, automated insertion 1200 begins. If the insertion mechanism (or other component of the insertion device) permits, the angle of insertion is adjusted according to the parameters set by the targeting system in step 1204. Then the end stop or insertion mechanism is adjusted to set the appropriate needle insertion depth at step 1206. When the insertion system is properly targeted, the insertion mechanism is actuated and the needle inserted at 1208. Next the needle is retracted and cannula advanced in step 1210.

Step 1212 is optionally performed by the insertion system or manually. In step 1212, the cannula is stabilised by suitable adhesive tape or the like. The needle, which is retracted into the insertion device and is then removed at step 1214 and the process can restart at 1216.

Returning to FIG. 11, after insertion, the imaging system can be used to gain visual confirmation of correct insertion of the cannula into the vessel at step 1124. Alternatively or additionally correct location of the cannula after insertion can be performed using impedance measurement. In this technique the needle is used as an electrode to measure the electrical impedance of the tissue. This can be performed in one embodiment using a needle having two electrodes formed concentrically about the needle shaft, but insulated between them. One electrode can be exposed at the needle tip and the other at a selected position to measure impedance over a predefined distance or location. Alternatively a single needle electrode, exposed at the tip can be used with a return electrode location on the skin surface.

In some embodiments, the body 102 of insertion device 100 and lower side of the imaging unit 104 that touches the patient's skin can be disposable. It can also be shaped to provide the final adhesive stabilisation to the cannula on final deployment. For example the lower side of the device can include an adhesive layer that sticks to the patient's skin.

This surface can also incorporate a layer formed of a conformant material, such as a disposable hydrogel pad that will conform to the patient anatomy and stabilise hand tremor and small movements. The hydrogel may additionally or alternatively server as an acoustic medium for coupling the ultrasound signal into the tissue. Additionally, or as an alternative, the surface could incorporate a sole plate or pad that is resiliently mounted, e.g. spring loaded, or pressure stabilised, so that the pressure of the device does not collapse the low pressure superficial veins that are being targeted. This constant pressure system could simply be the flat surface connected to a low force spring, where force remains relatively constant over a large deformation (<5 mm).

In some embodiments of the needle insertion device, a targeting system may not be included. Instead, the one or more parameters for the insertion mechanism can be predetermined in another way, e.g. set at manufacture, set manually, determined by a separate system or algorithmically determined with or without sensing. The parameters can still include any one or more of: a position of the end stop arrangement, length of stroke, position of the end of stroke (either linear or in three dimensions), angle of insertion; insertion location; time/speed of insertion, a needle trajectory, and so on.

With reference to FIG. 2b, the device 100 can be configured so that no imaging unit is present. The device 100 may be preloaded with a set of operational parameters for use on subjects or patients for which the parameters are suitable, e.g. a targeted group of patients. The targeted group can be, for example, men, women, children of certain age, patients that fit within a specific range of height or weight range etc. It will be appreciated that this embodiment can be particularly useful when the predetermined parameters would be suitable for a majority of the targeted group of users, because they all share similar physical characteristics.

The device 100 may be designed so that after a clinician finds an ideal insertion site, there is no further parameter adjustment required before using the device on the subject.

In another embodiment, the device 100 can be configured to allow one or more parameters to be set or adjusted manually by the clinician. For example, the depth of insertion may be mechanically adjusted by changing the position of the end stop arrangement 130, or by other suitable means.

In a further embodiment, the device 100 can include a targeting system that uses a targeting algorithm to determine the one or more parameters for a group of subjects. The device may allow a clinician to enter a subject's information, for example but not limited to a patient's age, gender, height, weight, ethnicity, and so on, and the targeting algorithm automatically generates a set of operational parameters based on the patient's information entered. The patient's information may be entered, e.g. via the touch display 106 or via other suitable means.

It will be understood that the invention disclosed and defined in this specification extends to all alternative combinations of two or more of the individual features mentioned or evident from the text or drawings. All of these different combinations constitute various alternative aspects of the invention.

1.-9. (canceled)

10. A needle insertion device configured to insert a needle into a lumen of a vessel of a subject, the insertion device comprising an insertion mechanism configured to insert the needle into the lumen of the vessel, in accordance with one or more predetermined parameters.

11. The needle insertion device of claim 10, wherein the predetermined parameters are selected and/or determined based on the subject's physical characteristics.

12. The needle insertion device of claim 10, wherein the insertion device further includes a targeting system configured to determine the one or more parameters.

13. The needle insertion device of claim 12, wherein the targeting system includes a targeting algorithm that receives as input one or more physical characteristics of the subject that the device is to be used on, and automatically determines the one or more parameters.

14. The needle insertion device of claim 12 wherein the targeting system includes a three dimensional imaging system; said targeting system being configured to determine one or more parameters for insertion of the needle into the lumen of the vessel.

15. The needle insertion device of claim 12, wherein the targeting system determines one or more of the following parameters for insertion of the needle: an insertion site on the tissue surface; a target position within the lumen of the vessel; a needle trajectory; angle of insertion; speed of insertion; and depth of insertion.

16. (canceled)

17. The needle insertion device of claim 10, wherein the insertion mechanism includes a carriage, which in use holds the needle and is translatable with respect to one or more guides.

18. The needle insertion device of claim 10, wherein the translation of the carriage is caused by an actuator.

19.-21. (canceled)

22. The needle insertion device of claim 18, wherein the insertion mechanism is arranged so that the carriage moves over a predetermined stroke.

23. The needle insertion device of claim 22, wherein the stroke can be fixed or variable.

24. The needle insertion device of claim 23, wherein the variable stroke is determined by the targeting mechanism prior to actuation of insertion.

25. The needle insertion device of claim 23, wherein when the fixed stroke is used, the end point of travel of the carriage is determined by adjusting the starting position of the carriage with respect to the lumen of the vessel, and the starting position of the carriage is determined by the targeting system.

26. The needle insertion device of claim 11, wherein the carriage includes a first needle carriage and a second carriage configured to carry a cannula arrangement during insertion.

27.-30. (canceled)

31. The needle insertion device of claim 14, wherein the three dimensional imaging system preferably includes at least two sensor systems.

32. The needle insertion device of claim 30, wherein the two sensor systems use different sensing modalities.

33. The needle insertion device of claim 32, wherein the sensing modalities include: any one or more of ultrasound; transillumination; near Infrared; visible or other EM radiation imaging; thermography; tactile/mechanical imaging; optical imaging including but not limited to photoacoustic/optoacoustic and optical coherence tomography; electrical imaging including but not, limited to electrical/RF tomography including MRI; radiation imaging, including but not limited, to Stereo X-Ray or CT scanning.

34. The needle insertion device of claim 14, wherein the targeting system includes a targeting algorithm that determines an ideal insertion site based on tissue properties and the patient's anatomy.

35.-36. (canceled)

37. The insertion device of claim 1, wherein the insertion mechanism is configured to insert the needle into the lumen of the vessel at high speed.

38. The insertion device of claim 18, wherein the high speed is more than 10 mm/s, 20 mm/s, 30 mm/s, 40 mm/s, 50 mm/s, 60 mm/s, 70 mm/s, 80 mm/s, 90 mm/s, 100 mm/s, or more than 200 mm/s, 250 mm/s, 300 mm/s, 400 mm/s, 500 mm/s, 600 mm/s, 700 mm/s, 800 mm/s, 900 mm/s, 1000 mm/s, or more than 1500 mm/s.

39. The insertion device of claim 38, wherein the high speed in less than 2000 mm/s.
